(19) United States
(12) Patent Application Publication
(10) Pub. No.: US 2023/0116973 A1
(43) Pub. Date: Apr. 20, 2023

(54) EMBOLIC PROTECTION DEVICE WITH DUAL-LAYER FILTER MESHES

(71) Applicant: SUZHOU ZENITH VASCULAR SCITECH LIMITED, SIP Suzhou (CN)

(72) Inventors: Bin YANG, SIP Suzhou (CN); Liqun JIAO, SIP Suzhou (CN); Liyou GUO, SIP Suzhou (CN); Jie XIA, SIP Suzhou (CN); Shuang LI, SIP Suzhou (CN)

(21) Appl. No.: 17/914,133

(22) PCT Filed: Apr. 6, 2021
(86) PCT No.: PCT/CN2021/085637

§ 371 Date: Sep. 23, 2022

(30) Foreign Application Priority Data
Nov. 10, 2020 (CN) 202011247654.6

(51) Int. Cl. A61F 2/01

(52) U.S. Cl. CPC A61F 2/0105; A61F 2/011; A61F 2210/0076; A61F 2250/0037; A61F 2250/0039; A61F 2230/0069; A61F 2230/0067; A61F 2002/016

(57) ABSTRACT

Provided is an embolic protection device with dual-layer filter meshes, including an elastic base frame, a first filter mesh and a second filter mesh. The two ends of the first filter mesh are the first open end and the first closed end respectively. The distance between the first closed end and the elastic base frame is greater than the distance between the first open end and the elastic base frame. The first open end is connected with the elastic base frame, and the first filter mesh is provided with a plurality of first filtering holes. The second filter mesh is provided with a plurality of second filtering holes.

This application claims priority to a Chinese patent application No. 202011247654.6 filed on Nov. 10, 2020, disclosure of which is incorporated herein by reference in its entirety.

### TECHNICAL FIELD

The application belongs to the medical device technology, for example, relates to an embolic protection device with dual-layer filter meshes.

### BACKGROUND

With improvement of living standards, the life span of the Chinese population has increased significantly, and the aging process is aggravated. The incidence of ischemic stroke is increasing and has become one of the major diseases endangering the lives of middle-aged and elderly people in China. Wherein, atherosclerotic carotid artery stenosis accounts for about 20% to 30% of ischemic stroke.

The intervention therapy of carotid artery stenosis is an important measure for preventing ischemic stroke. Conventional treatments for carotid artery stenosis are surgical operations such as carotid artery dissection, balloon dilatation, embolectomy and carotid artery stenting. Because of their features of the conventional treatments such as small surgical trauma, low anesthesia risk, fewer surgical contraindications and complications, the conventional treatments have been used as a primary treatment for carotid artery stenosis. However, during performance of the above-mentioned operations, thrombus or plaque often falls off, and the shed thrombus or plaque flows downstream of the blood vessel along with the blood flow, causing intracranial cerebral vascular embolism and thus leading to acute ischemic stroke.

A single-layer filter structure is generally adopted in the related art to filter thrombus or plaque, to prevent the thrombus or plaque from flowing downstream. However, the blood flow tends to be blocked when there are too many thrombi or plaques or there are large thrombi or plaques.

### SUMMARY

An embolic protection device with dual-layer filter meshes is provided, which can improve the effect of filtering thrombus or plaque.1

The following technical solutions are adopted in the application:

Provided is an embolic protection device with dual-layer filter meshes, including an elastic base frame, a first filter mesh and a second filter mesh.

The two ends of the first filter mesh are a first open end and a first closed end respectively. The distance between the first closed end and the elastic base frame is greater than the distance between the first open end and the elastic base frame. The first open end is connected with the elastic base frame, and the first filter mesh is provided with a plurality of first filtering holes.

The second filter mesh is provided with a plurality of second filtering holes. The second filter mesh is connected with the elastic base frame, and the second filter mesh is configured to sheathe an outside of the elastic base frame and an outside of the first filter mesh. The embolic protection device with dual-layer filter meshes is configured to allow the blood to flow downstream in the blood vessel through the elastic base frame, the first open end, the plurality of first filtering holes and the plurality of second filtering holes sequentially.

### BRIEF DESCRIPTION OF DRAWINGS

FIG. 1 is a schematic diagram of the embolic protection device with dual-layer filter meshes according to an embodiment of the application;

FIG. 2 is a schematic diagram of an embolic protection device with dual-layer filter meshes during usage according to the embodiment of the application;

FIG. 3 is a schematic diagram illustrating a connection between an elastic base frame and a first filter mesh of an embolic protection device with dual-layer filter meshes according to the embodiment of the application; and

FIG. 4 is a schematic diagram illustrating a structure of a second filter mesh of an embolic protection device with dual-layer filter meshes according to an embodiment of the application.

### REFERENCE LIST

100 embolic protection device with dual-layer filter meshes
10 elastic base frame
11 abutment portion
12 contracting end
20 first filter mesh
21 first open end
22 first closed end
30 second filter mesh
31 second open end
32 second closed end
33 filtering section
34 mounting section
51 large thrombus or plaque
52 small thrombus or plaque

### DETAILED DESCRIPTION

Detailed description of the present application is made below in conjunction with the figures and the embodiments. It can be understood that the embodiments described herein are only used to explain the application. It should also be noted that for the purpose of description, the figures only show some parts related to the application, but not the structure in whole.

In description of the present application, unless otherwise expressly specified and limited, the terms “connected”, “connect”, and “fixed” should be interpreted broadly. For example, it can be a fixed connection, a detachable connection, or a connection into a whole; a mechanical connection or an electrical connection; a direct connection or indirect connection through an intermediate medium, an internal connection of two elements or the interaction between two elements. For those of ordinary skill in the art, the meaning of the above terms in the application can be understood depending on the conditions.

In the application, unless otherwise expressly specified and limited, the case where the first feature is “on” or “under” the second feature may include the direct contact between the first and second features, and may also include other feature contact rather than direct contact between the first and second features. And the case where the first feature is “above”, “over”, and “on” the second feature indicates that the first feature is right and obliquely above the second feature, or only that the first feature is higher in level than the second feature. And the case where the first feature is “below”, “under”, and “underneath” the second feature indicates that the first feature is right and obliquely below the second feature, or only that the first feature is lower in level than the second feature.

In description of the present embodiment, “up”, “down”, “right” and other terms describing orientations or positions are based on the orientations or positions shown in the figures. They are used only for convenient description and simplified operation, rather than indicating or implying that the device or element referred to must stay in a specific position, or be constructed and operated in a specific orientation, so they should not be understood as a restriction over the application. Besides, the terms “first” and “second” are used only for the purpose of distinguishing in description, with no special meaning.

To facilitate description, the upstream and the downstream are defined by the flowing direction of blood in the blood vessel in the embodiment, and the blood flows from the upstream to the downstream.

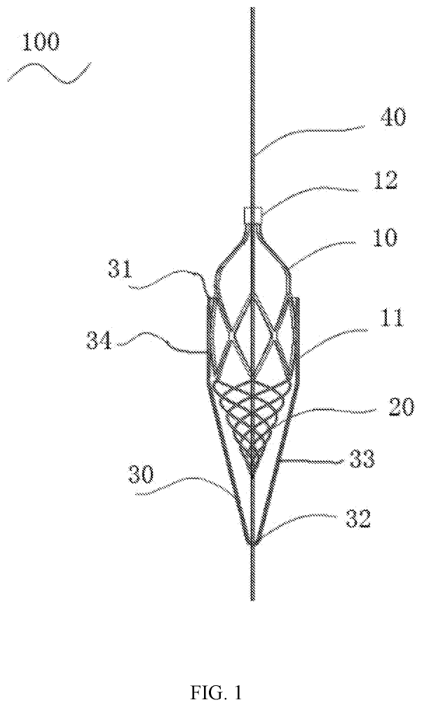

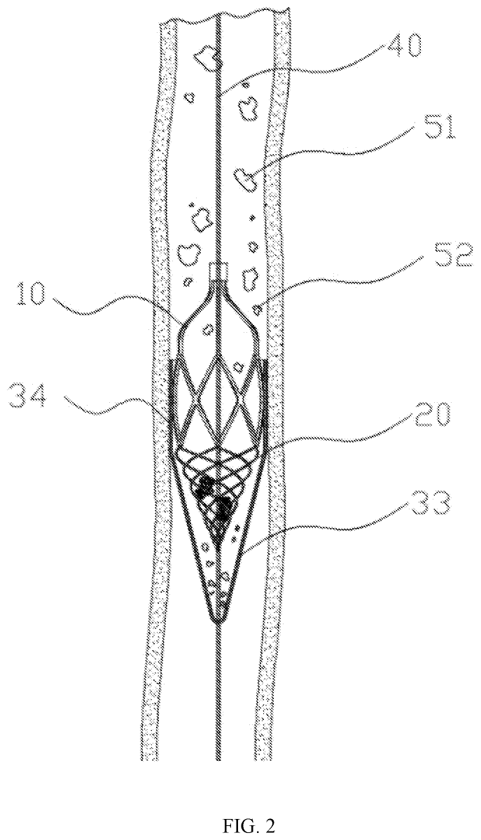

FIG. 1 is a schematic diagram of the embolic protection device 100 with dual-layer filter meshes according to an embodiment of the application. FIG. 2 is a schematic diagram of an embolic protection device with dual-layer filter meshes 100 during usage according to the embodiment of the application. As shown in FIGS. 1 and 2, the embodiment discloses an embolic protection device with dual-layer filter meshes 100. In the embodiments of the application, the embolic protection device with dual-layer filter meshes 100 may be an embolic distal-end-protection device with dual-layer filter meshes. The embolic protection device with dual-layer filter meshes 100 includes an elastic base frame 10, a first filter mesh 20 and a second filter mesh 30. The elastic base frame 10 may consist of a plurality of elastic rods which are staggered and hinged with each other, so that the elastic base frame 10 can freely expand and contract. One end of the elastic base frame 10 facing upstream forms a contracting end 12, and when the elastic base frame 10 expands, the other end of the elastic base frame 10 forms an open end. The elastic base frame 10 is configured to expand and contract, and using the contracting end 12 as the base point.

When the embolic protection device with dual-layer filter meshes 100 reaches the predetermined targeted position by the way of being connected, the embolic protection device with dual-layer filter meshes 100 is released. The elastic base frame 10 expands under the action of elasticity of the elastic base frame 10 to be fitted with and anchored on the inner wall of the blood vessel. A radial support force between the blood vessel and the elastic base frame 10 is produced, so that the elastic base frame 10 is limited by the blood vessel and thus the elastic base frame 10 is positioned at a predetermined position upstream in the blood vessel. The diameter of the blood vessel where the elastic base frame 10 is released is slightly smaller than the diameter of the blood vessel when the blood vessel is in a natural state and not bound by external forces, so the elastic base frame 10 acts a limited resilience force on the vessel wall and satisfies the force required for anchoring with no damage to the blood vessel.

The two ends of the first filter mesh 20 are the first open end 21 and the first closed end 22 respectively. The distance between the first closed end 22 and the elastic base frame 10 is greater than the distance between the first open end 21 and the elastic base frame 10. The first open end 21 is connected with the elastic base frame 10, and the first filter mesh 20 is provided with some first filtering holes. The second filter mesh 30 is provided with some second filtering holes. The second filter mesh 30 is connected with the elastic base frame 10. The second filter mesh 30 is configured to sheathe the outside of the elastic base frame 10 and the outside of the first filter mesh 20, so that the blood flows downstream in the blood vessel through the elastic base frame 10, the first open end 21, the first filtering holes and the second filtering holes sequentially during usage of the embolic protection device with dual-layer filter meshes 100.

The embolic protection device with dual-layer filter meshes 100 in this embodiment is provided with the first filter mesh 20 and the second filter mesh 30, where the second filter mesh 30 sheathes the outside of the first filter mesh 20, the second filter mesh 30 and the first filter mesh 20 form dual-layer filter meshes which can effectively filter the thrombus or plaque, and prevent the thrombus or plaque from flowing downstream in the blood vessel.

The first filter mesh 20 or the second filter mesh 30 in this embodiment can be connected with the elastic base frame 10 by bonding, hot melting, welding or sewing.

The first filter mesh 20 in this embodiment can be a polymer network structure. The first filter mesh 20 has a certain stiffness, so the shape and the mesh size of the first filter mesh 20 are not easy to change in the process of blood flowing and under the influence of thrombus or plaque. The diameter of the first filtering hole of the first filter mesh 20 ranges from 500 82 m to 1200 82 m, alternatively from 500 82 m to 600 82 m. For example, the diameter of the first filtering hole is 500 82 m, 600 82 m, or 1200 82 m. The first filter mesh 20 in this embodiment can also be a network structure woven with elastic metal wire or polymer wire.

The diameter of the second filtering hole of the second filter mesh 30 ranges 70 82 m to 150 82 m, alternatively from 80 82 m to 120 82 m. For example, diameter of the second filtering hole is 70 82 m, 80 82 m, 120 82 m or 150 82 m. Similarly, the second filter mesh 30 also has a certain stiffness, so the shape and the mesh size of the first filter mesh 20 are not easy to change in the process of blood flowing and under the influence of thrombosis or plaque. The second filter mesh 30 can be a network structure braided by elastic metal wire or polymer wire.

The elastic base frame 10 has a proper elastic force, so that the elastic base frame 10 can contract and drive the first filter mesh 20 and the second filter mesh 30 to contract when the embolic protection device with dual-layer filter meshes 100 is recovered. That is, the first filter mesh 20 and the second filter mesh 30 attached to the elastic base frame 10 contract as the diameter of the elastic base frame 10 becomes smaller and the elastic base frame 10 contracted. During usage, the embolic protection device with dual-layer filter meshes 100 is compressed until the diameter of the embolic protection device with dual-layer filter meshes 100 is minimum. The diameter of the embolic protection device with dual-layer filter meshes 100 is released when entering the blood vessel and reaching the predetermined position. Then, the elastic base frame 10 rebounds until the diameter of the embolic protection device with dual-layer filter meshes 100 is equivalent to the diameter of the blood vessel, so that the embolic protection device with dual-layer filter meshes is fitted with the blood vessel wall.

The first filter mesh 20 and the second filter mesh 30 in this application also opens when the elastic base frame 10 expands, to filter thrombus or plaque. The diameters of the first and second filtering holes mentioned below refer to the diameters when they are opened. The structures of the elastic base frame 10, the first filter mesh 20 and the second filter mesh 30 in this application all refer to the structures expanded.

Alternatively, the second filter mesh 30 and the first filter mesh 20 are spaced apart from each other. When the first filter mesh 20 and the second filter mesh 30 expands, the first filter mesh 20 and the second filter mesh 30 are spaced apart from each other and not in touch with each other. At least the actual filtering positions of the first filter mesh 20 and the second filter mesh 30 in this embodiment are spaced apart from each other. The dual-layer filter meshes including filter meshes spaced apart from each other are arranged in this embodiment, and the two filter meshes have filtering holes with different diameters, to filter thrombus or plaque having different sizes. Therefore, thrombus or plaque can be distributed in the axial direction of the embolic protection device with dual-layer filter meshes 100, and can be prevented from gathering, it ensures that blood flows normally, and blood blockage, perfusion or turbulence are prevented. And it also facilitates final recovery of the embolic protection device with dual-layer filter meshes 100. When there are thrombi or plaques with a large particle size captured by the first filter mesh 20, the first filter mesh 20 can cut the large thrombi or plaques into small pieces under the impact of blood flow. The small thrombi or plaques are finally captured by the second filter mesh 30.

In terms of a single-layer filter mesh, the captured thrombi or plaques are accumulated on the single-layer filter mesh, which tends to result in thrombus or plaque accumulation and affect normally flowing of blood during operation. Besides, since thrombus or plaque is gathered on the single-layer filter mesh, the local radial diameter is too large. It is often hard to recover the embolic protection device with dual-layer filter meshes 100 after operation. Therefore, the dual-layer filter meshes including filter meshes spaced apart from each other are arranged in this embodiment, to disperse the thrombi or plaques along with the trend of the blood vessel, which does not cause the local radial diameter to be too large and ensures that it is easy to recover the embolic protection device with dual-layer filter meshes 100.

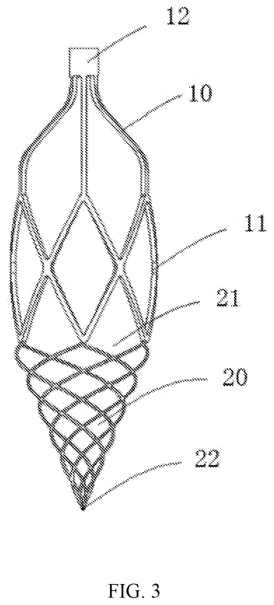

FIG. 3 is a schematic diagram illustrating a connection between an elastic base frame 10 and a first filter mesh 20 of an embolic protection device with dual-layer filter meshes 100 according to the embodiment of the application. As shown in FIGS. 1 and 3, alternatively, the opening areas of the first filtering holes gradually decrease in the direction from the first open end 21 to the first closed end 22, which is helpful to improve the effect of the first filter mesh 20 in filtering thrombus or plaque.

Alternatively, the first filter mesh 20 is a conical structure, and the diameter of the first filter mesh 20 gradually decreases in the direction from the first open end 21 to the first closed end 22, which is helpful to improve the effect of the first filter mesh 20 in filtering thrombus or plaque.

Alternatively, the opening area of each of the second filtering holes is smaller than the opening area of each of the first filtering holes. In this embodiment, the large thrombus or plaque 51 is filtered through the first filter mesh 20; the small thrombus or plaque 52 enters the space between the first filter mesh 20 and the second filter mesh 30 through the first filtering hole, and stays in the space between the first filter mesh 20 and the second filter mesh 30 after being filtered by the second filtering holes of the second filter mesh 30. Thrombi or plaques are filtered by layers sequentially in this embodiment with a satisfactory filtering effect.

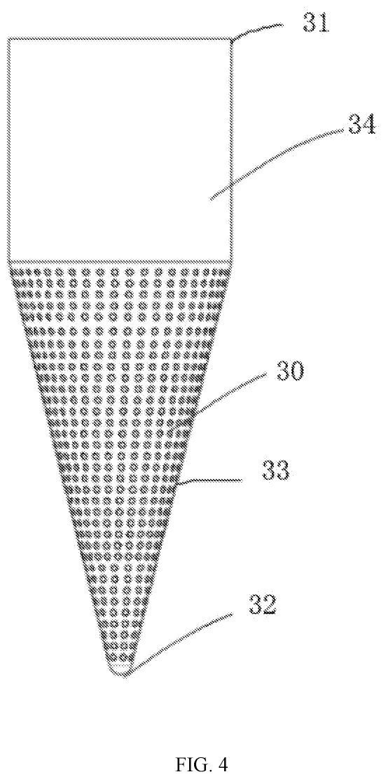

FIG. 4 is a schematic diagram illustrating a structure of a second filter mesh 30 of an embolic protection device with dual-layer filter meshes 100 according to an embodiment of the application. As shown in FIGS. 1 and 4, alternatively, the second filter mesh 30 includes a filtering section 33, and the second filtering holes are distributed in the filtering section 33; the filtering section 33 is a conical structure, and the diameter of the filtering section 33 gradually decreases in the direction away from the elastic base frame 10, which is helpful to improve the effect of the second filter mesh 30 in filtering thrombus or plaque.

Alternatively, the filtering section 33 is configured to sheathe the first filter mesh 20, and the opening of the filtering section 33 is parallel to the first open end 21, so that the first filter mesh 20 and the second filter mesh 30 in this embodiment filter thrombi or plaques at the same time; the first filter mesh 20 filters large thrombus or plaque 51, and the second filter mesh 30 filters small thrombus or plaque 52, thus filtering more fully. The opening of the filtering section 33 herein refers to the opening on the end of the filtering section 33 facing downstream. The other end of the filtering section 33 opposite to the opening forms the second closed end 32 of the entire second filter mesh 30.

As shown in FIGS. 2 and 4, alternatively, the second filter mesh 30 includes a filtering section 33 and a mounting section 34 connected with the filtering section 33; the mounting section 34 is a cylindrical structure, and the mounting section 34 is configured to sheathe and connected to the outside of the elastic base frame 10. Structure of the mounting section 34 in this embodiment facilitates the connection between the second filter mesh 30 and the elastic base frame 10, and makes it easy to sheathe the second filter mesh 30 the outside of the first filter mesh 20. The end of the mounting section 34 facing upstream forms a second open end 31 of the entire second filter mesh 30.

As shown in FIGS. 1, 2 and 4, alternatively, the elastic base frame 10 includes an abutment portion 11, the outer wall of the abutment portion 11 protrudes toward the inner wall of the blood vessel, and the mounting section 34 is configured to sheathe the periphery of the abutment portion 11 and is bound together with the abutment portion 11. There is a mounting section 34 between the abutment portion 11 of the elastic base frame 10 and the inner wall of the blood vessel in this embodiment. The outer surface of the mounting section 34 better fits the local structure of the blood vessel, which is more helpful to protect the blood vessel. The mounting section 34 in this embodiment also has certain elasticity. The abutment portion 11 combined with the mounting section 34 can better abut against the blood vessel to be positioned at the predetermined position, and the abutment portion 11 does not move during operation.

Alternatively, the embolic protection device with dual-layer filter meshes 100 further includes a guide wire 40. The guide wire 40 is connected to one end of the elastic base frame 10. In an embodiment, the guide wire 40 is connected to the contracting end 12.

The guide wire 40 is used to deliver the embolic protection device with dual-layer filter meshes 100 to a predetermined site in the vessel, for example, to deliver the embolic protection device with dual-layer filter meshes 100 to the target site of lesion. The guide wire 40 may be a device for subsequent interventional surgery operations such as a guide wire, a catheter, an imaging catheter, a balloon, a thrombectomy catheter, a suction catheter, a stent conveyor, and a recovery catheter. The guide wire 40 can be made of biocompatible metal or polymer materials, for example, metals such as nickel-titanium alloy, stainless steel, or cobalt-chrome alloy, or polymer materials such as nylon, PI, or PET. The outer diameter of the guide wire 40 generally ranges from 0.010 to 0.038 Inch, alternatively is 0.014 Inch.

The elastic base frame 10, the first filter mesh 20 and the second filter mesh 30 deform when being folded, thus leading to turbulence, swirling or disturbance of blood flow during recovery of the embolic protection device with dual-layer filter meshes 100. The structures of the first filter mesh 20 and the second filter mesh 30 in this embodiment make it difficult for the captured thrombus or plaque to break away from the embolic protection device with dual-layer filter meshes 100 during the recovery of the embolic protection device with dual-layer filter meshes 100.

In other embodiments, the first filter mesh 20 and the second filter mesh 30 may also be in a shape of a windsock.

Alternatively, when the first filter mesh 20 is a metal filter mesh, for example, when the first filter mesh 20 is a nickel-titanium alloy filter mesh or a cobalt-chrome alloy filter mesh or a stainless steel filter mesh, the first filter mesh 20 is convenient to connect with the metal elastic base frame 10, for example, it can be integrated with the elastic base frame 10.

In the present application, the first filter mesh and the second filter mesh configured to sheathe the first filter mesh are provided so that dual-layer filter meshes are formed. With this configuration, thrombus or plaque can be effectively filtered and the thrombus or the plaque can be prevented from flowing downstream in the blood vessel. Further, since the thrombus is distributed on both the first filter mesh and the second filter mesh when the whole embolic protection device with dual-layer filter meshes is recovered, it is convenient to recover the embolic protection device with dual-layer filter meshes, and the thrombus is prevented from escaping.

1. An embolic protection device with dual-layer filter meshes, comprising:
an elastic base frame;
a first filter mesh, wherein two ends of the first filter mesh are a first open end and a first closed end respectively, a distance between the first closed end and the elastic base frame is greater than a distance between the first open end and the elastic base frame, the first open end is connected with the elastic base frame, and the first filter mesh is provided with a plurality of first filtering holes; and
a second filter mesh, provided with a plurality of second filtering holes, wherein the second filter mesh is connected with the elastic base frame, and the second filter mesh is configured to sheathe an outside of the elastic base frame and an outside of the first filter mesh, and the embolic protection device with dual-layer filter meshes is configured to allow blood to flow downstream in a blood vessel through the elastic base frame, the first open end, the plurality of first filtering holes and the plurality of second filtering holes sequentially.

2. The embolic protection device with dual-layer filter meshes according to claim 1, wherein the second filter mesh and the first filter mesh are spaced apart from each other.

3. The embolic protection device with dual-layer filter meshes according to claim 1, wherein opening areas of the plurality of first filtering holes gradually decrease in a direction from the first open end to the first closed end.

4. The embolic protection device with dual-layer filter meshes according to claim 3, wherein the first filter mesh is a conical structure, and diameters of the first filter meshes gradually decrease in the direction from the first open end to the first closed end.

5. The embolic protection device with dual-layer filter meshes according to claim 1, wherein an opening area of each of the plurality of the second filtering holes is smaller than an opening area of each of the plurality of the first filtering holes.

6. The embolic protection device with dual-layer filter meshes according to claim 5, wherein the second filter mesh comprises a filtering section, wherein the plurality of second filtering holes are distributed in the filtering section, the filtering section is a conical structure, and a diameter of the filtering section gradually decreases in a direction away from the elastic base frame.

7. The embolic protection device with dual-layer filter meshes according to claim 6, wherein the filtering section is configured to sheathe the first filter mesh, and an opening of the filtering section is parallel to the first open end.

8. The embolic protection device with dual-layer filter meshes according to claim 1, wherein the second filter mesh comprises a filtering section and a mounting section connected to the filtering section, wherein the mounting section is a cylindrical structure, and the mounting section is configured to sheathe and is connected to the outside of the elastic base frame.

9. The embolic protection device with dual-layer filter meshes according to claim 8, wherein the elastic base frame comprises an abutment portion, wherein an outer wall of the abutment portion is configured to protrude towards an inner wall of the blood vessel, the mounting section is configured to sheathe a periphery of the abutment portion and is bound with the abutment portion.

10. The embolic protection device with dual-layer filter meshes according to claim 1, further comprising a guide wire connected with an end of the elastic base frame.

11. The embolic protection device with dual-layer filter meshes according to claim 1, wherein the first filter mesh is a metal filter mesh.

12. The embolic protection device with dual-layer filter meshes according to claim 1, wherein the second filter mesh is a polymer filter mesh.

13. The embolic protection device with dual-layer filter meshes according to claim 11, wherein the first filter mesh is a nickel-titanium alloy filter mesh, or a cobalt-chrome alloy filter mesh, or a stainless steel filter mesh.

14. The embolic protection device with dual-layer filter meshes according to claim 2, wherein an opening area of each of the plurality of the second filtering holes is smaller than an opening area of each of the plurality of the first filtering holes.

15. The embolic protection device with dual-layer filter meshes according to claim 3, wherein an opening area of each of the plurality of the second filtering holes is smaller than an opening area of each of the plurality of the first filtering holes.

16. The embolic protection device with dual-layer filter meshes according to claim 4, wherein an opening area of each of the plurality of the second filtering holes is smaller than an opening area of each of the plurality of the first filtering holes.

17. The embolic protection device with dual-layer filter meshes according to claim 11, wherein the second filter mesh is a polymer filter mesh.
